(19) United States
(12) Patent Application Publication
(10) Pub. No.: US 2009/0138040 A1
(43) Pub. Date: May 28, 2009

(54) CRANIUM APPARATUS

(76) Inventor: AFSHIN AL FALLAH, San Diego, CA (US)

Correspondence Address: WOOD AND EISENBERG, PLLC, 6911 RICHMOND HIGHWAY, SUITE 403, Alexandria, VA, 22306, US

(21) Appl. No.: 12/325,109

(22) Filed: Nov. 28, 2008

Related U.S. Application Data
(60) Provisional application No. 60/990,617, filed on Nov. 28, 2007.

(51) Int. Cl. A61F 5/00

(52) U.S. Cl. 606/204.15

(57) ABSTRACT

A cranial apparatus for applying pressure and/or decompression to a patient's skull, the cranial apparatus includes: a means for providing left and right reversible inflation against the left and right sides of a patient's skull; a means for providing opposing left and right counter forces in the x-axis; a means for providing downward pressure in the y-plane against the top of a patient's skull; a means for providing a counter-force in an upwards direction in the y-axis; and a means for providing working fluid to the means for providing left and right reversible inflation against the left and right sides of a patient's skull.

### CROSS-REFERENCE TO RELATED APPLICATIONS

This application claims the benefit of priority from U.S. Provisional Patent Application Ser. No. 60/990,617 (filed Nov. 28, 2007) the contents of which are incorporated herein by reference in their entirety.

### STATEMENT REGARDING FEDERALLY SPONSORED RESEARCH OR DEVELOPMENT

Not Applicable.

### FIELD OF THE INVENTION

This invention relates to an apparatus for compressing cranial bones. More specifically, the invention is directed to an apparatus for controlled compression and decompression of cranial bones located on the top and opposite sides of the cranium.

### BACKGROUND OF THE INVENTION

As noted in U.S. patent publication number 20060161200, it is thought that the majority of cells in the central nervous systems (CNS) of mammals obtain at least a portion of their nutrients via cerebral-spinal fluid (CSF). Using humans as an example, inside a person's cranium, there is a membrane called the dura mater that adheres to the bone. CSF circulates, at least in part, between layers of the meninges and is found between the dura mater and the next membrane. Similarly, in the spine, there is the bone of the vertebra, a layer of membrane within the vertebra and then CSF before the next layer of membrane. In the cranium, there are a total of three layers of membrane between the bones of the cranium and the brain. Likewise, in the spinal cord, there are three layers of membrane between the bone material of the vertebra and the CNS tissue of the spinal cord. It is thought that the CSF lubricates these membranes and by a process called diffusion delivers nutrients to the nerve tissue of the brain and spinal cord.

Various symptoms may arise if the circulation of CSF is interrupted or otherwise compromised. Persons with compromised circulation can experience symptoms such as fatigue, lack of mental focus, and lack of energy. Increasingly sedentary life styles associated with contemporary urban living can have a negative impact on normal circulation of CSF. Many occupations involve spending several hours sitting, for example, in front of computer screens. Jobs involving physical exertion are becoming less common in light of our increasing dependence on machines.

### SUMMARY

A cranial apparatus for applying pressure and decompression to a patient's skull, the cranial apparatus includes: a means for providing left and right reversible inflation against the left and right sides of a patient's skull; a means for providing opposing left and right counter forces in the x-axis; a means for providing downward pressure in the y-plane against the top of a patient's skull; a means for providing a counter-force in an upwards direction in the y-axis; and a means for providing working fluid to the means for providing left and right reversible inflation against the left and right sides of a patient's skull.

### BRIEF DESCRIPTION OF THE DRAWINGS

FIG. 1 shows a perspective environmental view of a first embodiment of the cranial apparatus according to the present invention.

FIG. 2 shows the cranial apparatus of FIG. 1 partially disassembled.

FIGS. 3A and 3B show a table (Table 1).

FIG. 4 shows a perspective view of a left L-bracket according to the present invention.

FIG. 5 shows a top planar view of the left L-bracket of FIG. 4.

FIG. 6 shows an optional left L-bracket according to the present invention.

FIG. 7 shows a perspective view of a right L-bracket according to the present invention.

FIG. 8 shows a top planar view of the right L-bracket of FIG. 7.

FIG. 9 shows an optional right R-bracket according to the present invention.

FIGS. 10 through 12 show various views of a toothed gear according to the present invention.

FIGS. 13 and 14 show cutaway views of a hand operated syringe pump according to the present invention.

FIG. 15 shows a cranial device with a roller-type peristaltic pump according to the present invention.

FIG. 16 shows a cranial device with a roller-type peristaltic pump and a pair of two-way valves according to the present invention.

FIG. 17 shows a cranial device with a roller-type peristaltic pump and a manual switch according to the present invention.

FIG. 18 shows the component parts that make up an intra-oral counter-force mechanism according to the present invention.

FIGS. 19 and 20 respectively show top and bottom views of a top bladder plate according to the present invention.

FIGS. 21 and 22 respectively show top and bottom views of a top plate according to the present invention.

FIG. 23 shows opposite side views of a left bladder plate according to the present invention.

FIG. 24 shows opposite side views of left side bladder according to the present invention.

FIG. 25 shows opposite side views of the top bladder according to the present invention.

FIGS. 26 through 29 respectively show bottom, perspective front, perspective rear, and top views of the cranial device according to the present invention.

FIG. 30 shows a view of the cranial device fitted with a chinstrap according to one embodiment of the invention.

FIG. 31 shows a rear view of the cranial device of FIG. 30.

FIG. 32 shows a view of the cranial device fitted with a chinstrap and a rear neck strap according to one embodiment of the invention.

FIG. 33 shows a rear view of the cranial device of FIG. 32.

FIG. 34 shows a view of the cranial device fitted with a chinstrap and a rear-neck strap according to one embodiment of the invention.

FIG. 35 shows a rear view of the cranial device of FIG. 34.

### DETAILED DESCRIPTION OF THE INVENTION

This invention relates to a cranial apparatus for controlled cyclic compression and decompression of cranial bones achieved by applying pressure cyclically to the cranium bones of a patient's skull. More specifically, the invention is directed to a cranial apparatus for controlled compression and decompression of cranial bones located on the top and opposite sides of a patient's cranium. The cranial apparatus of the present invention is denoted generally by the numeric label “100”. The terms “device” and “apparatus” are regarded hereinafter as equivalent terms.

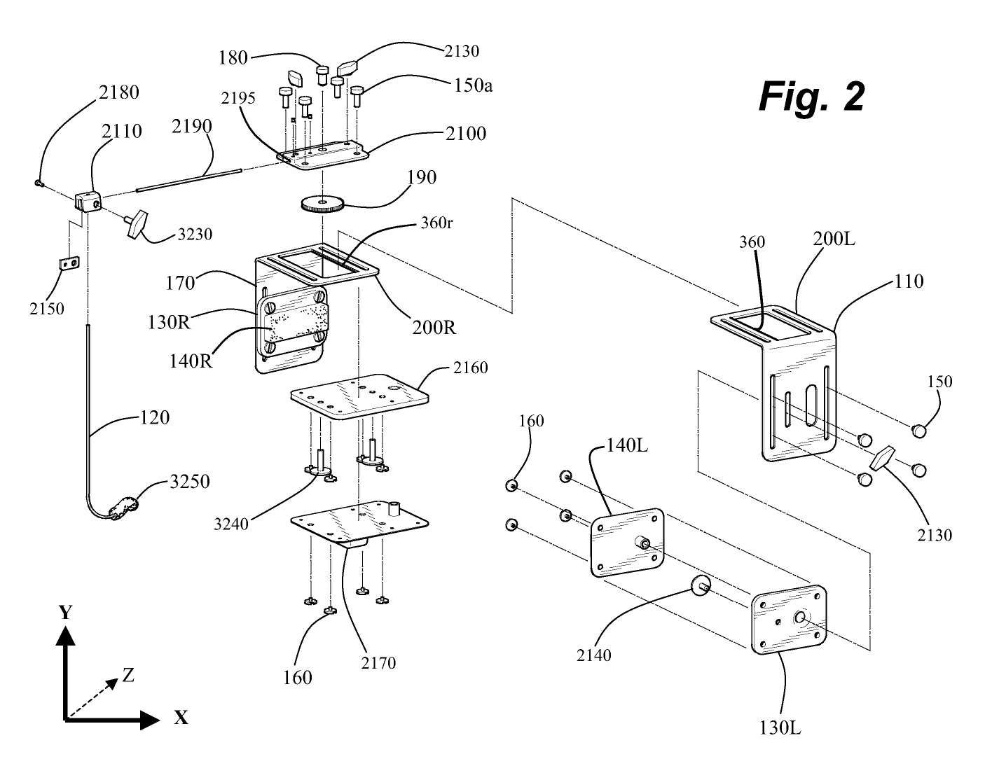

For purposes of description herein, the terms “upper,” “lower,” “right,” “left,” “rear,” “front,” “vertical,” “horizontal,” and derivatives thereof shall relate to the invention as oriented in FIG. 2. More specifically, for the purposes of description the horizontal corresponds with the widely known x-axis, the vertical with the widely known y-axis and depth with the widely known z-axis. However, it is to be understood that the invention may assume various orientations, except where expressly specified to the contrary.

It should be understood that for the purposes of this paper the terms “pouches” and “bladders” are regarded as equivalent terms. The bladders inflate and deflate when working fluid WF is pumped into and out of the bladders, respectively. In one embodiment, the top bladder inflates as the left and right side bladders simultaneously deflate whereupon the top bladder deflates as the left and right side bladders simultaneously inflate.

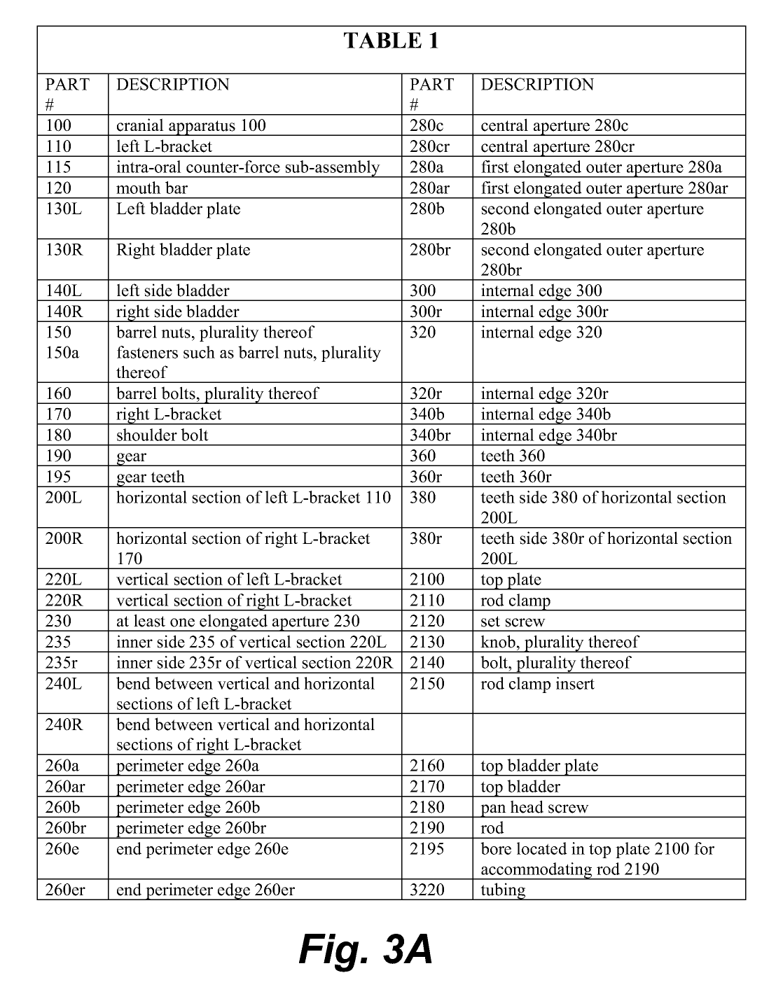

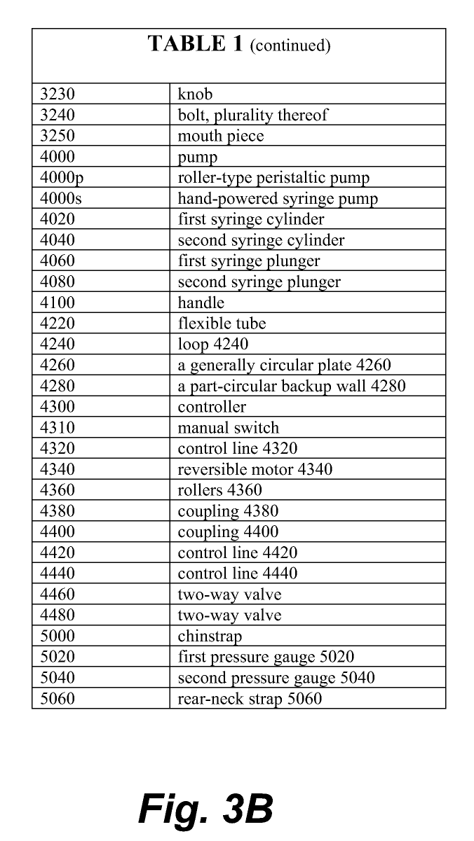

A summary of the component parts that make up the various embodiments of the cranial apparatus 100 are listed in Table 1 (see FIGS. 3A and 3B). It will be understood by a person of ordinary skill in the art that some component parts that make up the cranial apparatus 100 may be varied in many ways by a routineer in the applicable arts. Such variations are not to be regarded as a departure from the spirit and scope of the invention and all such modifications are intended to be included with the scope of the claims.

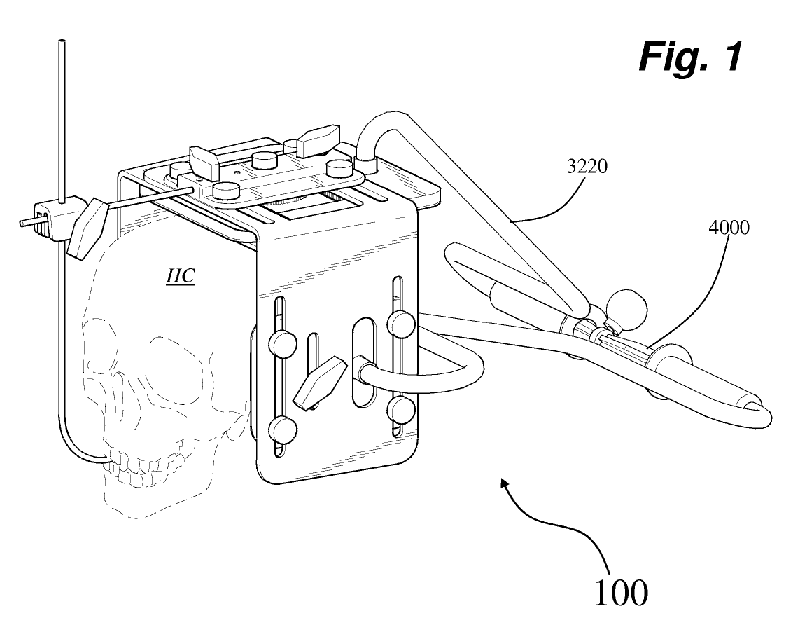

Referring to the Figures in general of which FIG. 1 shows a perspective environmental view of a first embodiment of the cranial apparatus 100. The cranial apparatus 100 is shown fitted around a patient's cranium, and more specifically around a human cranium HC. The human cranium HC is shown in outline. The cranial apparatus 100 is designed to apply cycles of controlled compression and decompression to the cranial bones on the top, left-side and right-side of a patient's cranium.

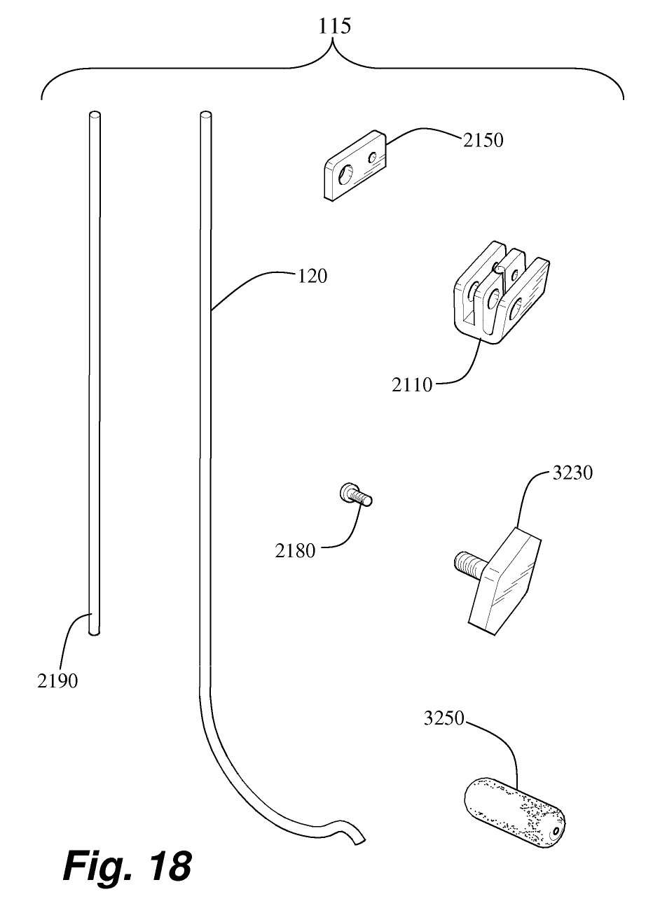

FIG. 2 shows the cranial apparatus 100 partially disassembled (tubing 3220 and pump system 4000 not shown). Items shown include left and right L-brackets 110 and 170, respectively; at least one top bladder 2170, at least one left side bladder 140L and at least one right side bladder 140R; and an intra-oral counter-force sub-assembly 115 (see FIG. 18) for providing, when required, a counter force in the y-axis with respect to the top bladder 2170. While only one each of the top 2170, left 140L and right 140R side bladders are shown in FIG. 2 it should be understood that at least one top bladder, at least one left side bladder and at least one right side bladder are used in the cranial apparatus 100. Thus, there could be more than one top, left side and right side bladders used in the cranial device 100.

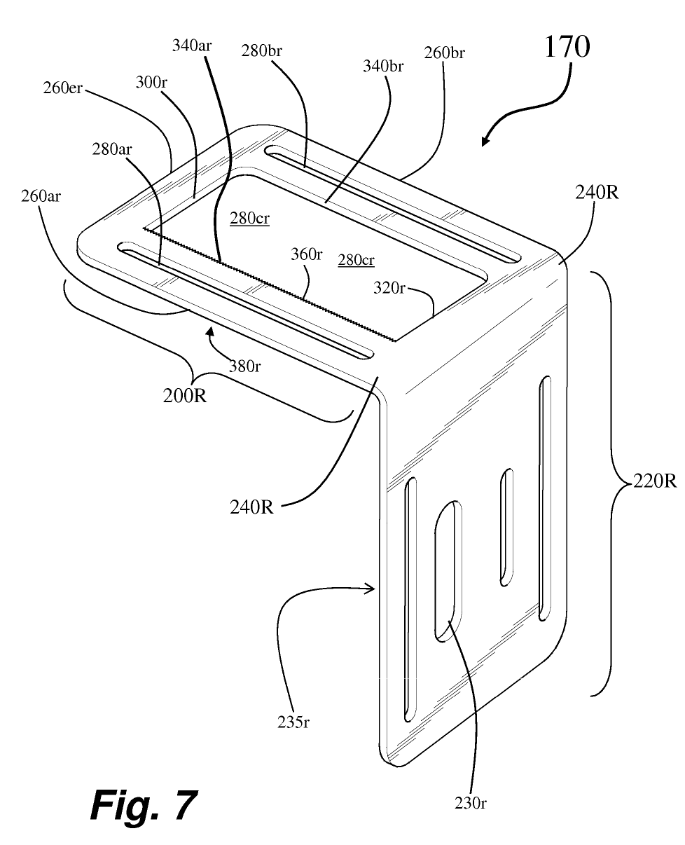

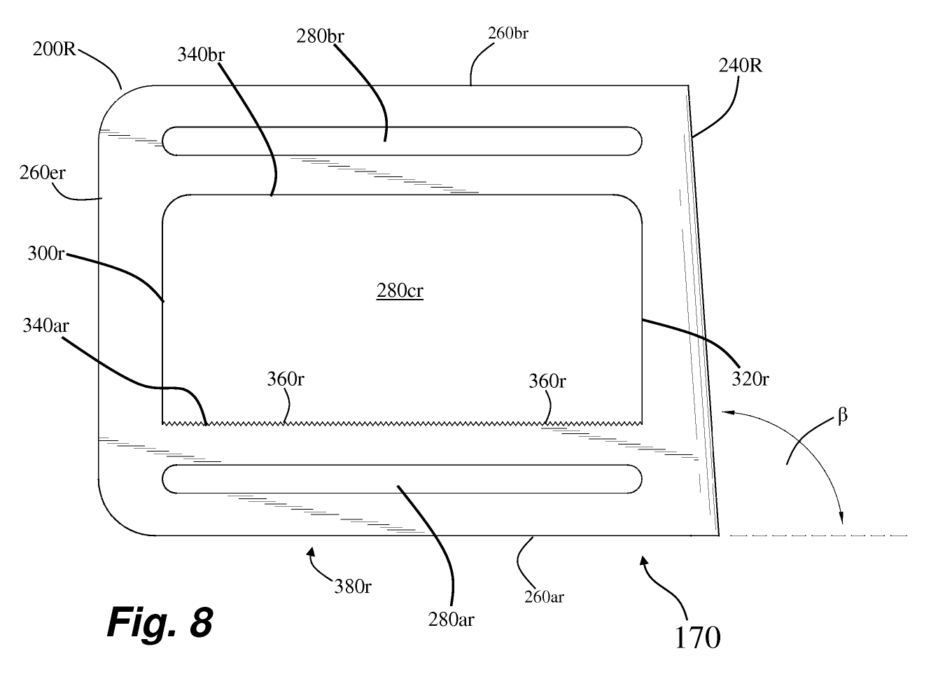

Upon reversible inflation of the left side bladder 140L the left side bladder 140L provides an inwards force in the horizontal plane along the x-axis directed into the left side of a patient's cranium and the corresponding counter force is provided by the vertical section 220R of right L-bracket 170 (shown in FIGS. 7 and 8).

Upon reversible inflation of the right side bladder 140R the right side bladder 140R provides an inwards force in the horizontal plane along the x-axis directed into the right side of a patient's cranium and the corresponding counter force is provided by the vertical section 220L.

The intra-oral counter-force sub-assembly 115 (see FIG. 18) is connected at its top end via rod 2190 to top plate 2100, and terminates at its bottom end with mouth piece 3250. The intra-oral counter-force sub-assembly 115 is made up of several components including rod 2190, rod clamp 2110, and at its bottom end mouth piece 3250. During normal use of the cranium apparatus 100, the mouth piece 3250 is placed inside the mouth of a patient and is adjusted such that the mouth piece 3250 is placed proximate to a patient's maxilla to rest up against the roof of a patient's mouth to provide, when required, an appropriate counter-force to the opposite direction force from the top bladder 2170 upon inflation of the top bladder 2170 against the top of a patient's skull, wherein the terms “skull” and “cranium” are regarded herein as equivalent terms.

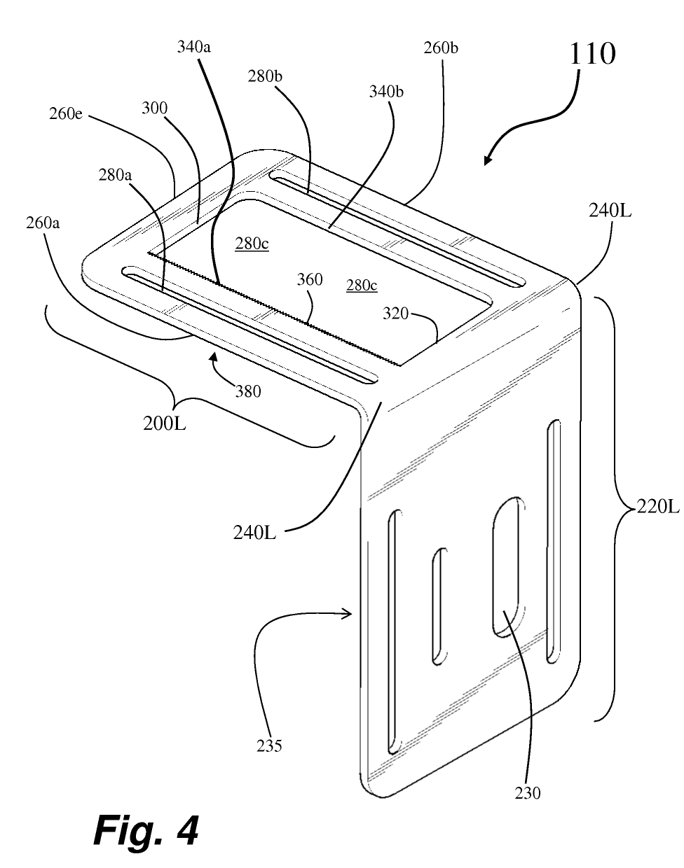

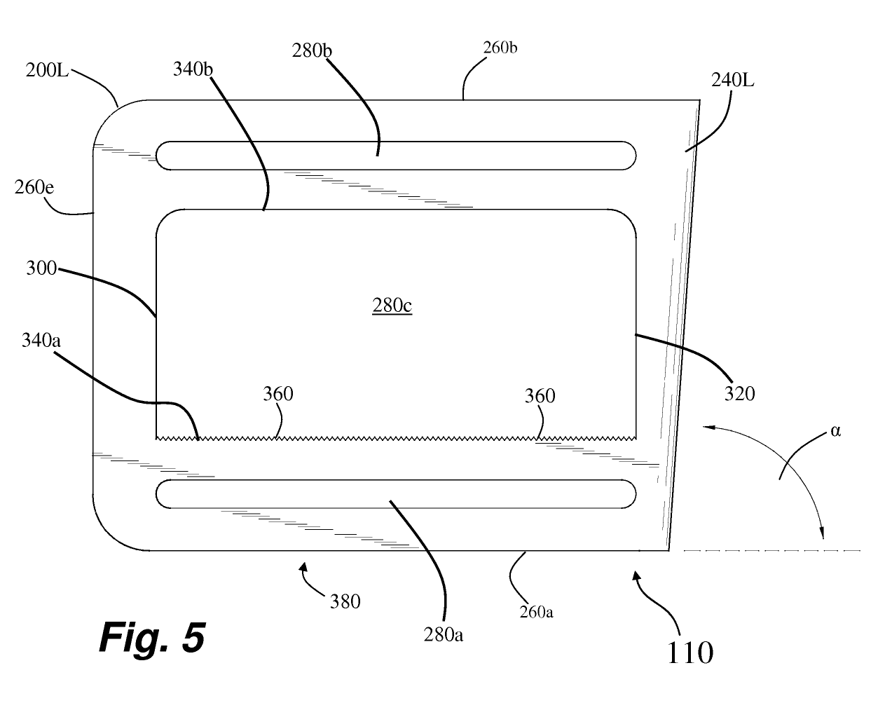

With reference to FIGS. 4 and 5, the left L-bracket 110 comprises a horizontal section 200L and a vertical section 220L. The vertical section 220L includes at least one elongated aperture 230 to permit adjustment of the left side bladder 140L in the vertical plane with respect to the inner side 235 of vertical section 220L. The left L-bracket 110 defines a bend 240L between the horizontal and vertical sections 200L and 220L. The horizontal section 200L defines three perimeter side edges made up of a two opposite parallel perimeter edges 260a and 260b and an end perimeter edge 260e. The horizontal section 200L further comprises a central aperture 280c, and a first and second elongated outer apertures 280a and 280b one each side of the central aperture 280c and running parallel and adjacent to opposite parallel edges 260a and 260b, respectively. The central aperture 280c is of generally rectangular shape and is located between the first and second apertures 280a and 280b and extends most of the length of the horizontal section 200L.

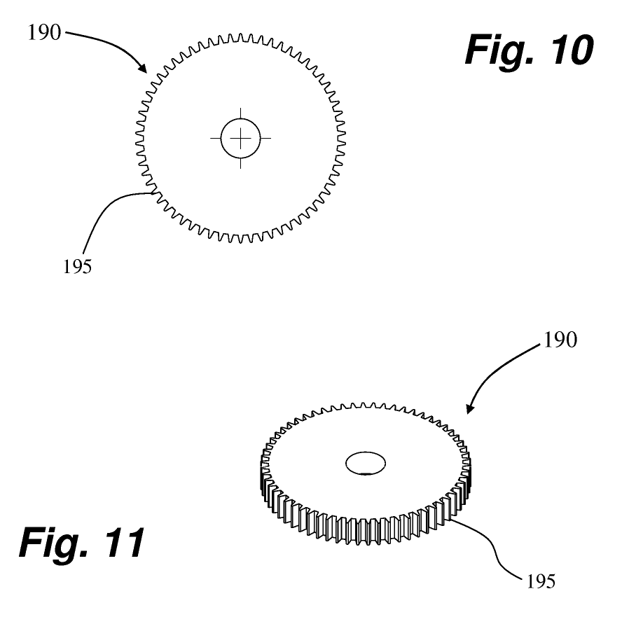

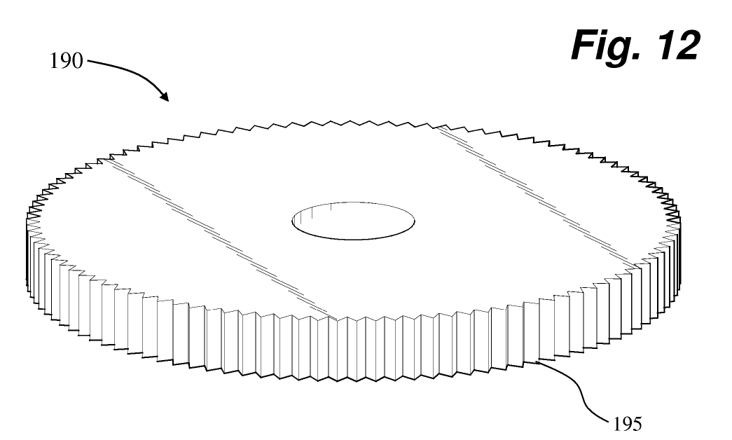

The length of the central aperture 280c is defined by opposite internal edges 300 and 320, and the width of the central aperture 280c is defined by opposite internal edges 340a and 340b. At least part of internal edge 340a is lined with a row of teeth 360. The teeth 360 complement gear teeth 195 on gear 190, wherein teeth 195 cooperatively engage teeth 360 (and teeth 360r on the right L-bracket 170) such that turning a shoulder bolt 180 (shown in FIG. 2) causes the horizontal section 200L, and hence left L-bracket 110, to adjust position to allow an operator, such as a medical practitioner or nurse, to fit the cranial apparatus 100 comfortably to a patient's head. Close up perspective views of gear 190 is shown in FIGS. 10 through 12.

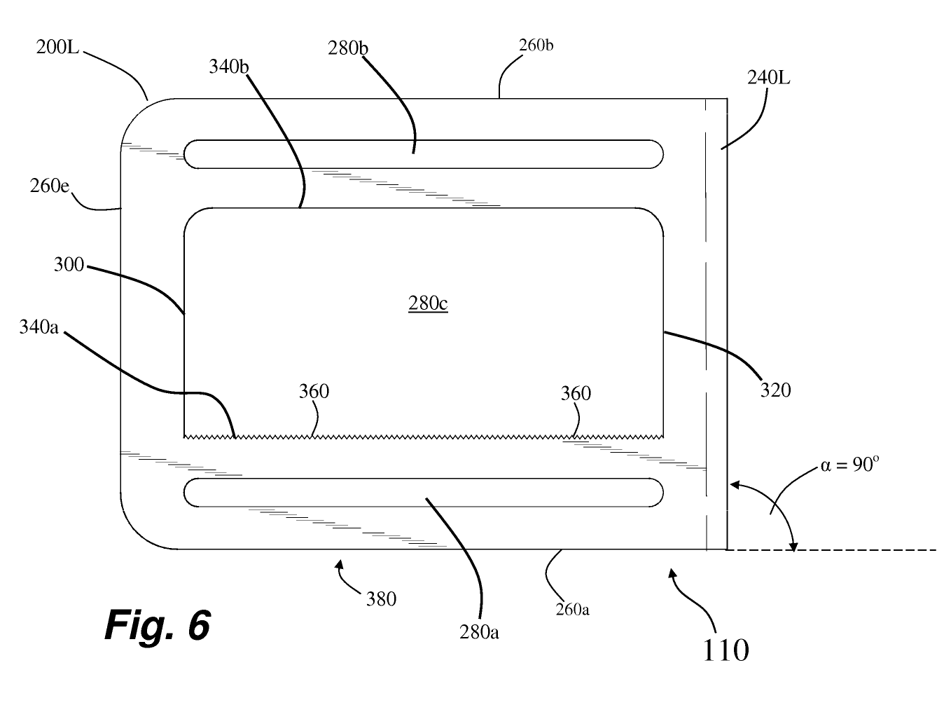

The row of teeth 360 defines teeth side 380 of horizontal section 200L. With respect to the teeth side 380 of the horizontal section 200L, the bend 240L has an external angle alpha (α) of between 80° and 90° (see FIG. 5), and more preferably an external angle alpha (α) of between 84° and 88°. In one embodiment the external angle alpha (α) is 86°±1°. Optionally, the external angle alpha (α) is 90° (see FIG. 6).

With reference to FIGS. 7 and 8, the right L-bracket 170 comprises a horizontal section 200R and a vertical section 220R. The vertical section 220R includes at least one elongated aperture 230r to permit adjustment of the left side bladder 140R (not shown in FIG. 7 or 8, but shown, for example, in FIG. 2) in the vertical plane with respect to the inside side 235r of vertical section 220R. The right L-bracket 170 defines a bend 240R between the horizontal and vertical sections 200R and 220R. The horizontal section 200R defines three perimeter side edges made up of a two opposite parallel perimeter edges 260ar and 260br and an end perimeter edge 260er. The horizontal section 200R further comprises a central aperture 280cr, and a first and second elongated outer apertures 280ar and 280br one each side of the central aperture 280cr and running parallel and adjacent to opposite parallel edges 260ar and 260br, respectively. The central aperture 280cr is of generally rectangular shape and is located between the first and second apertures 280ar and 280br and extends most of the length of the horizontal section 200R.

The length of the central aperture 280cr is defined by opposite internal edges 300r and 320r, and the width of the central aperture 280cr is defined by opposite internal edges 340ar and 340br. At least part of internal edge 340ar is lined with a row of teeth 360r. The teeth 360r complement gear teeth 195 on gear 190, wherein teeth 195 cooperatively engage teeth 360r (and teeth 360 on the left L-bracket 110) such that turning shoulder bolt 180 (shown in FIG. 2) causes the horizontal section 200R (and 200L, but in opposite way), and hence right L-bracket 170, to adjust position to allow an operator, such as a medical practitioner or nurse, to fit the cranial apparatus 100 comfortably to a patient's head.

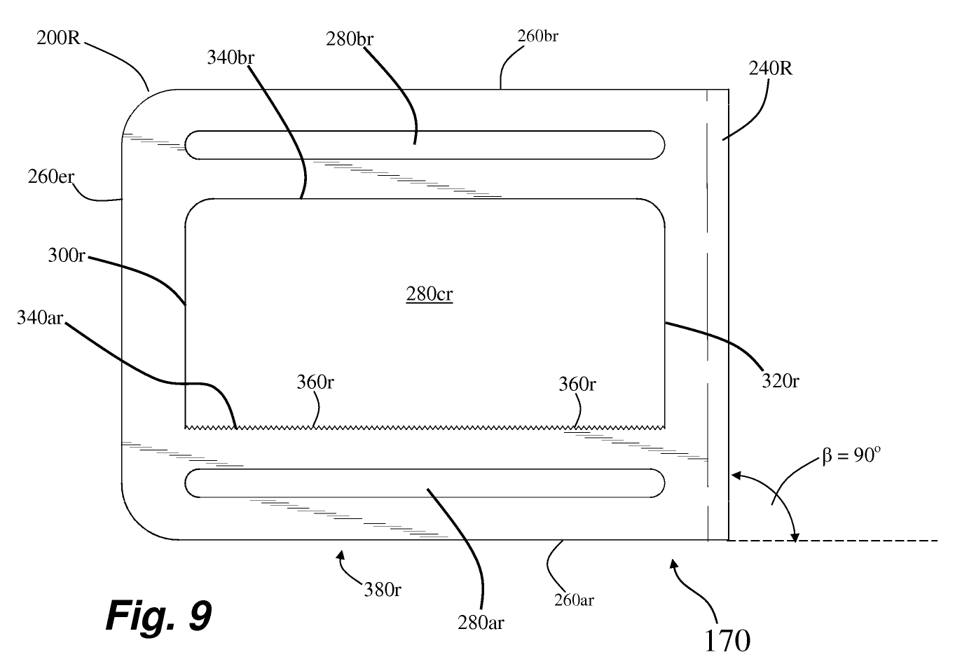

The row of teeth 360r defines teeth side 380r of horizontal section 200R. With respect to the teeth side 380r of the horizontal section 200R, the bend 240R has an external angle beta (β) of between 90° and 100° (see FIG. 8), and more preferably an external angle beta (β) of between 92° and 98°. In one embodiment the external angle beta (β) is 95°±2°. In one embodiment beta (β) is 94°. Optionally, the external angle beta (β) is 90° (see FIG. 9).

During normal use of the cranial apparatus 100, teeth rows 360 and 360r are in a trans configuration, i.e., teeth rows 360 and 360r are located across from each other, i.e., teeth rows 360 and 360r are not in a cis configuration, i.e., not on the same side. Thus, gear teeth 195 cooperatively engage teeth rows 360 and 360r to make position adjustments with respect to left and right L-brackets 110 and 170 to allow an operator to make adjustments to the cranial apparatus 100 when fitting the device to a patient's head.

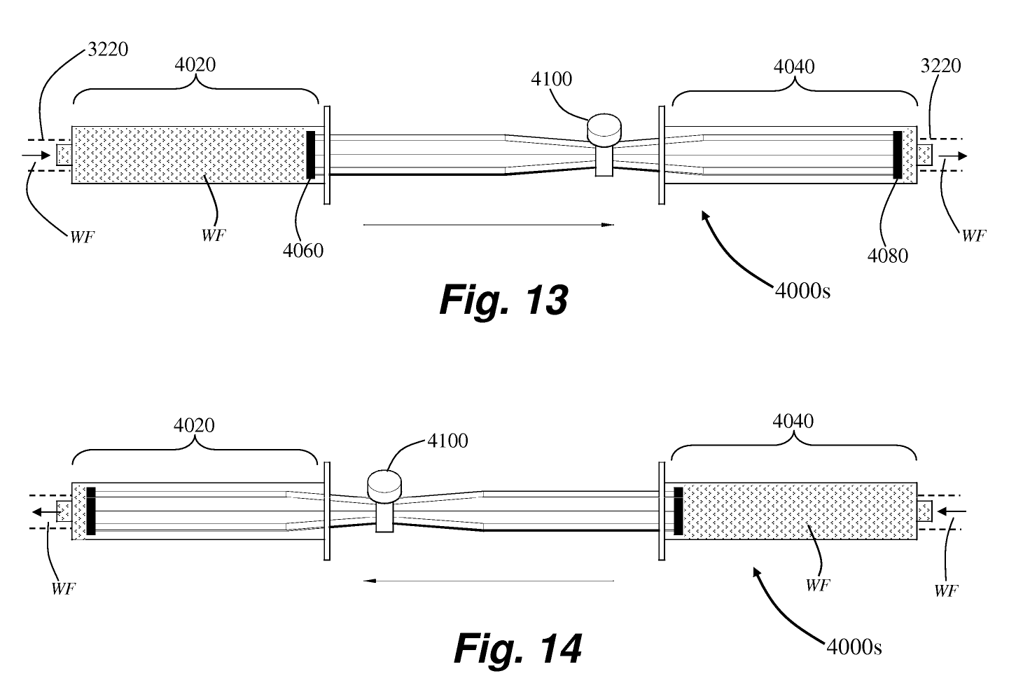

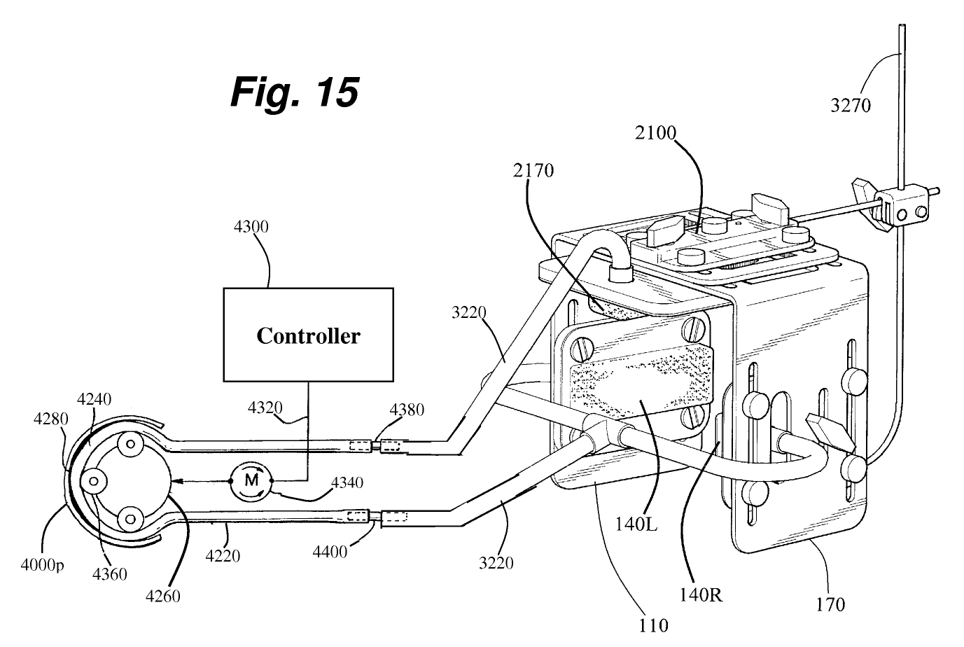

The cranial apparatus 100 works in conjunction with a pump system 4000 (example of which is shown in FIG. 1), which can take any suitable form of pump such as a hand operated syringe pump 4000s (see FIGS. 13 and 14) or electricity driven pump, e.g., a peristaltic pump. One type of suitable pump is a reversible pump such as, but not limited to: a roller-type peristaltic pump 4000p (see, e.g., FIG. 15). A roller-type peristaltic pump in combination with a reversible motor is described in U.S. Pat. No. 4,755,168; U.S. Pat. No. 4,755,168 is incorporated herein by reference in its entirety.

A suitable pump is operably connected to the bladders by means of tubing 3220. Working fluid WF used to reversibly inflate bladders (such as bladders labeled as 140L, 140R and 2170 in FIG. 2) can take any suitable form such as a gas or liquid. Suitable liquids include, but are not limited to, salt water with sufficient amount of salt to restrict unwanted microbial growth or biological fouling in the tubing 3220 and/or pump system 4000. The tubing 3220 can be any suitable diameter and may be transparent or opaque. If the tubing 3220 is selected to be opaque this would help prevent light entering the tubing 3220 and thus further help to avoid growth of light requiring organisms such as algae or other microbes with light absorbing chlorophyll or other light absorbing medium.

In one non-limiting embodiment, the cranial the pump 4000 is constructed as illustrated in FIGS. 13 and 14. In this embodiment the cranial pump 4000 is in the form of a hand-powered double-ended-syringe pump 4000s made up of horizontally opposed first and second syringe cylinders 4020 and 4040 with first and second syringe plungers 4060 and 4080 arranged in a horizontally opposed arrangement. A handle 4100 is used to operate the hand-powered double-ended-syringe 4000s. During normal operation the opposite ends of the hand-powered double-ended-syringe 4000s are connected to tubing 3220. When the handle 4100 is pushed to the right (see FIG. 13), working fluid WF enters the first syringe cylinder 4020 from tubing 3220 while working fluid is simultaneously ejected from second syringe cylinder 4040 into tubing 3220. When the handle 4100 is pushed to the left (see FIG. 14), working fluid WF is ejected from the first syringe cylinder 4020 while simultaneously it is caused to enter the second syringe cylinder 4040 from tubing 3220.

The hand-powered double-ended-syringe 4000s thus drives working fluid WF in both directions; move handle 4100 one way to reversibly inflate the top bladder 2170 while simultaneously reversibly deflating left and right side bladders 140L and 140R; move handle 4100 the other way to reversibly deflate the top bladder 2170 while simultaneously reversibly inflating left and right side bladders 140L and 140R. The hand-powered double-ended-syringe 4000s can therefore be regarded as a non-limiting example of a reversible flow pump system.

The handle 4100 can be moved in opposite directions in synchrony with a patient's breathing; alternatively, the handle 4100 is moved in opposite directions as desired preferably under direction or supervision of a medical professional such as a suitably qualified and experienced medical doctor or nurse.

Referring to FIG. 15 in which a roller-type peristaltic pump 4000p is shown. The roller-type peristaltic pump 12 includes a flexible tube 4220 of a resilient polymer material. Part of flexible tube 4220 is formed into a loop 4240. A generally circular plate 4260 includes a plurality of rollers spaced at a common radius about its circumference. A part-circular backup wall 4280 surrounds a portion of loop 4240. A controller 4300 produces control signals for application on a control line 4320 to reversible motor 4340. Responsive to its control signals reversible motor 4340 rotates plate 4260 in a selected direction, and at a selected speed. As plate 4260 rotates, rollers 4360 squeeze loop 4240 against part-circular backup wall 4280 thus urging fluid flow through loop 4240 in the direction of rotation of plate 4260. Controller 4300 can comprise any suitable microprocessor integrated circuit including a process unit and memory. The memory may include random access memory (RAM), read only memory (ROM), erasable programmable ROM (EPROM) and/or a data storage component such as a hard drive.

Still referring to FIG. 15, tubing 3220 is coupled to the ends of flexible tube 4220 by means of couplings 4380 and 4400. Depending on the direction of rotation of plate 4260 working fluid is directed either to the top bladder 2170 (and hence from the left and right side bladders 140L and 140R) or working fluid is directed to the left and right side bladders 140L and 140R (and hence from top bladder 2170). Thus, either the top bladder 2170 is inflating (in which case side bladders 140L and 140R are being deflated) or the top bladder 2170 is being deflated while side bladders 140L and 140R are simultaneously being inflated.

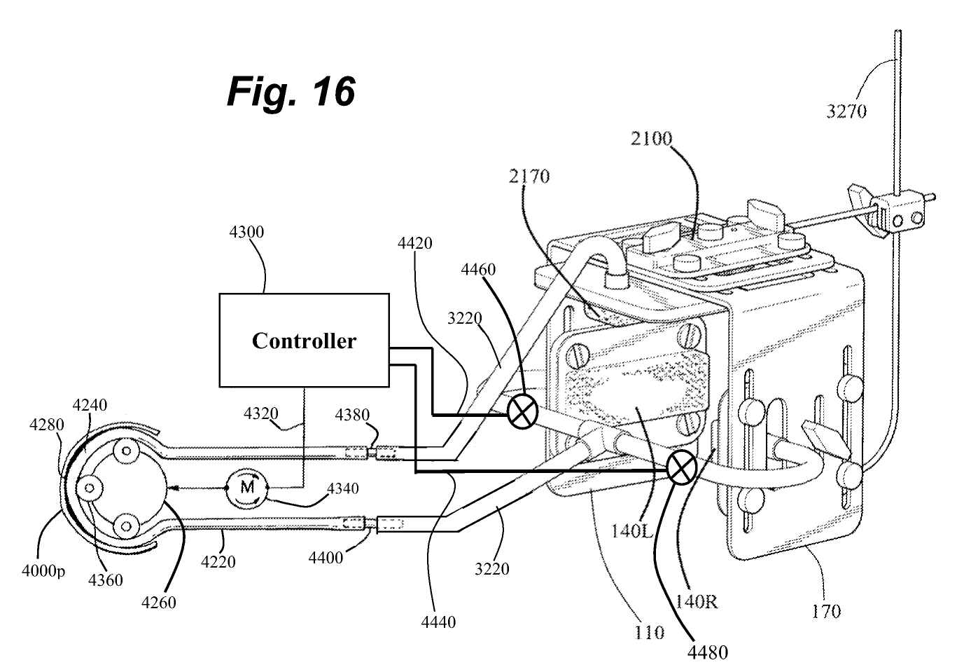

Referring now to FIG. 16, which shows a diagram similar to FIG. 15 except that the drawing includes two additional control lines 4420 and 4440, and two two-way valves 4460 and 4480. More specifically, the controller 4300 now controls both the reversible motor 4340 and the two-way valves 4460 and 4480. In this embodiment the controller 4300 is configured to direct working fluid to the top bladder 2170 or to either or both of the side bladders 140L and 140R. Thus, the bladders 2170, 140L and 140R can be inflated or deflated in any desired sequence.

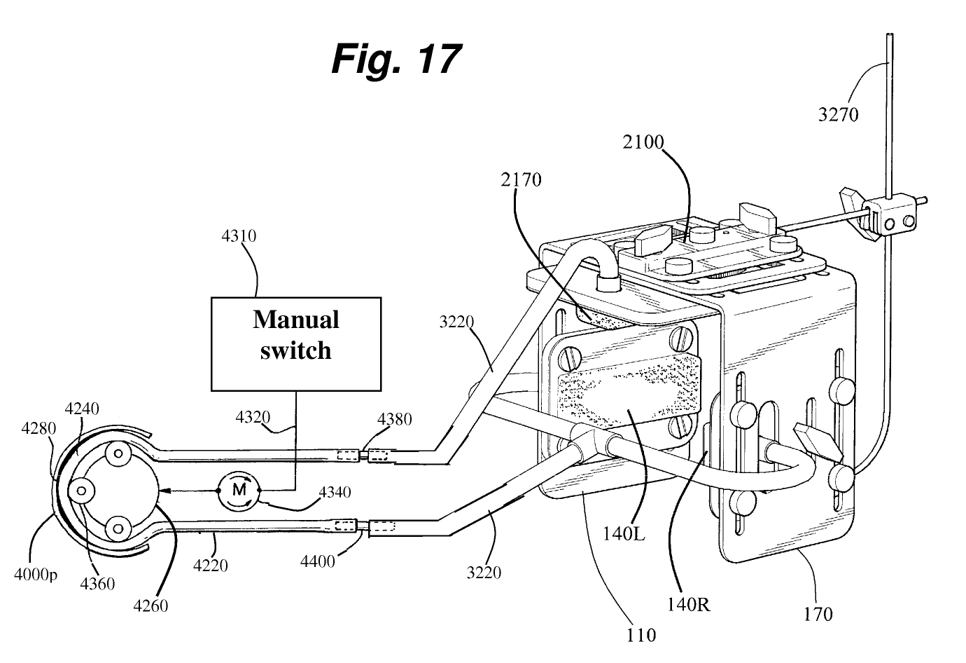

Referring now to FIG. 17, which shows a diagram similar to FIG. 15 except that the controller 4300 is replaced with a manual switch 4310, which sends control signals to the reversible motor 4340. The manual switch 4310 enables the patient or a medical professional to operate the reversible motor 4340 and hence inflate the top bladder 2170 (while deflating the side bladders 140L and 140R) or deflate the top bladder 2170 while inflating the side bladders 140L and 140R. The patient can operate the manual switch 4310 in synchrony with their breathing and hence inflate and deflate the bladders in synchrony with their breathing.

The left and right side bladders 140L and 140R, exerts sufficient force to cause inward movement of the cranial bones on the left and right sides of a patient's cranium. The top bladder 2170 exerts sufficient downwards force in the y-axis to cause inward movement of the cranial bones on the top sides of a patient's cranium. Thus, cycles of compression and decompression can be concentrated on at least one cranium bone during the operation of the cranial apparatus 100, e.g., on temporal bones, parietal bones, sphenoid bones, frontal bones and the occipital bone.

In one non-limiting embodiment, as the side bladders 140L and 140R are simultaneously pressurized (i.e., inflated), the top bladder 2710 is depressurized (i.e., deflated). Conversely, as the side bladders 140L and 140R start to depressurize the top bladder 2170 pressurizes against top of the patient's cranium (e.g., over the top of parietal bones and the frontal bone).

When the top bladder 2170 pressurizes, a vertical counter force with respect to an inflating top bladder 2170 is provided by any suitable means such as an intra-oral mouthpiece 3250, which during normal use of the cranial apparatus 100 fits inside a patient's mouth. The intra-oral mouthpiece 3250 is connected to a mouth bar 120 and thence to a rod 2190. The rod 2190 is attached to a top plate 2100 (see FIG. 2). The horizontal inward counter forces in the horizontal x-plane with respect to inflating left and right bladders 140L and 140R are provided by vertical sections 220L and 220R of left and right brackets 110 and 170, respectively.

A predetermined time interval can be imposed for each cycle of inflation/deflation. For example, the top bladder inflates while the left and right side bladders simultaneously deflate, wherein the top bladder remains inflated and the side bladders remain deflated for a predetermined time interval T₁. After time T₁ the top bladder deflates while the left and right bladders simultaneously inflate, wherein the top bladder remains deflated and the side bladders remain inflated for a predetermined time interval T₂. The time periods T₁ and T₂ can be equal in duration or differ in duration, e.g., T₁=T₂ or T₁>T₂ or T₁<T₂ (i.e., T₁≧T₂ or T₁<T₂).

Alternatively, only one bladder is cycled through rounds of inflation and deflation. For example, only the top bladder is cycled through periods of inflation and deflation. Alternatively, only the left side bladder is cycled through periods of inflation and deflation. Alternatively, only the right side bladder is cycled through periods of inflation and deflation.

FIG. 18 shows the component parts that make up the intra-oral counter-force sub-assembly 115, which are separately listed in Table 1 (see FIGS. 3A and 3B). The mouth piece 3250 provides a vertical counter force (with respect to an inflating top bladder 2170) against the palatal area (roof of the mouth).

The cranial apparatus 100 of the present invention is expected to ease pressure or stress between the cranial bones along the cranial sutures, and create a pumping action by flexure of cranial bones to improve the circulation of the Cerebral Spinal Fluids (CSF). More specifically, the bladders 140L, 140R and 2170 are believed to cause flexure of cranial bones to alter the intra-cranial environment, wherein movement of the CSF is artificially induced by causing controlled flexure of selected cranial bones.

The pressurization of the at least one top bladder 2170 puts direct force on the sagittal suture, the lamboid suture and the coronal suture. The inventor believes that the cranial sutures flex 0-2 mm under low to moderate force (force of 1-5 ounce/square inch). The compression force can pressurize the superior sagittal sinus (which is filled with CSF). The inventor believes that the pressurization of the superior sagital sinus helps to enhance the drainage of the CSF in into the venous system.

The inventor believes that the side bladders pressurization via contact on the sutures between the Temporal Bones, Frontal Bones, Sphenoid Bones and the Parietal Bones creates a transient increase in the intra cranial pressure. The inventor believes that the transient increase in the CSF aids pumping and the drainage of the CSF into the venous system.

In one variation of the invention, the practitioner may skip pressurizing the side bladders 140L and 140R altogether and only use the at least one top bladder 2170 in conjunction with the intraoral device 115 and/or the chinstrap 5000. This can be done in cyclical manner with or with out synchrony with the patient's breathing.

The cranial apparatus 100 of the present invention creates a pumping action to improve the circulation of the Cerebral Spinal Fluids (CSF) by inducing movements of the cranial bones. It is thought that some of these movements occur along the cranial sutures joining adjacent cranial bones. More specifically, the cranial apparatus 100 causes or otherwise induces movements of the cranial bones wherein at least one cranium bone, but not all the cranium bones simultaneously, is moved inwards (i.e. towards the brain) in response to a directly applied compression load. The amount of movement is sufficient to cause improved pumping/circulation of CSF around the brain and spinal column without harming the brain. The apparatus also helps to relieve stress, when present, along the cranial sutures that interconnect adjacent cranial bones.

It is believed by the inventor that the cranial apparatus 100 is able to exploit some movement of the cranial sutures that interconnect adjacent cranium bones that make up the skull. The use of the apparatus 100 in this manner contrasts with current medical opinion which believes the sutures are “immovable fibrous joints” (see, e.g., Gray's Anatomy for Students, published 2005, page 752, ISBN 0-443-06612-4).

It should be understood that the pump shown in FIG. 15 is shown merely for purposes of illustration, i.e., any suitable pump of any suitable design can be used and is not limited in any way to the specific hand-operated opposed syringe cylinder pump shown in FIG. 15.

It should be understood that any dimensions shown in the attached Figures are merely for purposes of illustration and can be varied without detracting from the spirit of the present invention. In particular, with respect to the above description and attached drawings, it is to be realized that the optimum dimensional relationships for the parts of the present invention may include variations in size, materials, shape, form, function and manner of operation.

In one embodiment, the side bladders 140L and 140R, when pressurized with working fluid WF, put mild to moderate force on temporal bones, parietal bones, sphenoid bones, frontal bones and the occipital bone. As the side bladders 140L and 140R are getting pressurized, the top bladder 2170 is simultaneously depressurizing. As the depressurization of side bladders 140L and 140R starts, the top bladder 2170 is pressurizing the top of the cranium (over the top of parietal bones and the frontal bone). When the top bladder 2170 pressurizes, the counter force is provided by any suitable device such as, but not limited to, the intra-oral counter-force sub-assembly 115. The intra-oral counter-force sub-assembly 115 provides the counter force against the palatal area, i.e., the roof of the patient's mouth. Ideally the pressurization/depressurization can be paired with inhalation and exhalation in different combinations, but it should be understood that the apparatus 100 can be used independent of the breathing patterns of the patient to which the apparatus 100 is fitted.

The apparatus of the present invention 100 is expected to provide a patient at least one of the following benefits: (1) release excess pressure, if present, between the cranial bones along the cranial sutures; (2) have a massaging effect on muscles with their insertions and/or origins on the cranial bones; and (3) create a pumping effect to improve the circulation of the Cerebral Spinal Fluids (CSF).

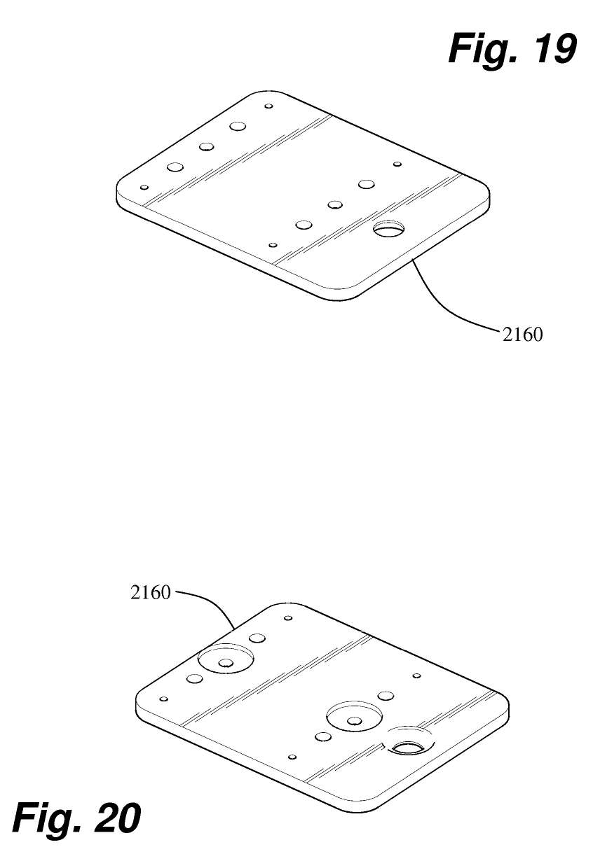

FIGS. 19 and 20 respectively show top and bottom views of a top bladder plate 2160. The top bladder plate 2160 fits between the top bladder 2170 and L-brackets 140L and 140R as shown in the exploded view of FIG. 2.

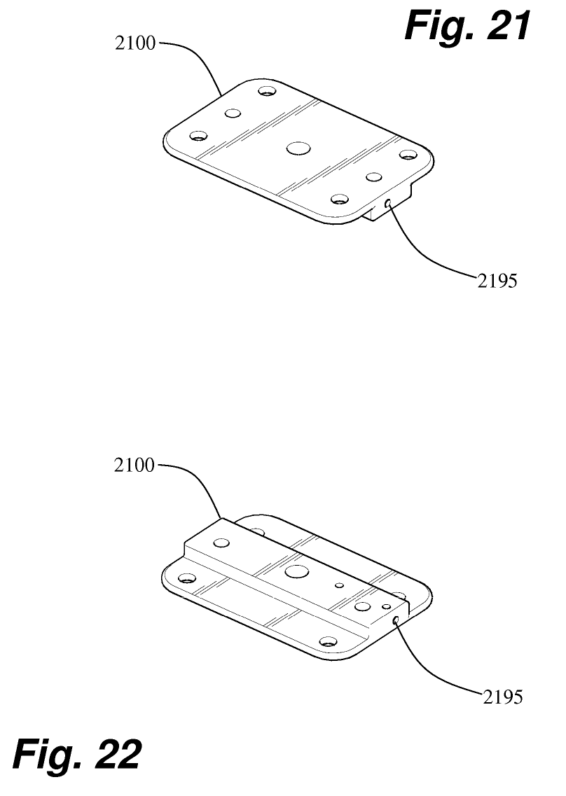

FIGS. 21 and 22 respectively show top and bottom views of a top plate 2100. The top plate 2100 fits above the L-brackets 140L and 140R as shown in the exploded view of FIG. 2. The top plate 2100 includes a bore 2195 for receiving rod 2190; rod 2190 forms part of the intra-oral counter-force sub-assembly 115 (see FIG. 18). The top plate 2100 aligns the gear 190 between the top plate 2100 and the top bladder plate 2160. The gear 190 is situated inside central apertures 280c and 280cr with its gear teeth 195 in contact with teeth rows teeth 360 and 360r. As shown in the exploded view of FIG. 2, shoulder bolt 180 operates through the top plate 2100 to turn the gear 190 and hence serve to adjust the relative positions of the right and left side L-brackets 110 and 170, respectively.

During normal operation of the cranial apparatus 100, the horizontal sections 200L and 200R are sandwiched between the top plate 2100 and top bladder plate 2160. The horizontal section 200L rests on top of horizontal section 200R or horizontal section 200R rests on top of horizontal section 200L. The gear 190 (and gear teeth 195) has sufficient depth to ensure good contact with both rows of teeth 360 and 360r. The top plate 2100 is fastened to the top bladder plate 2160 by fasteners 150a which are disposed through outer apertures 280a, 280ar, 280b and 280br. Outer apertures 280a and 280ar are respectively shown in FIGS. 4 and 7; and outer apertures 280b and 280br are respectively shown in FIGS. 4 and 7.

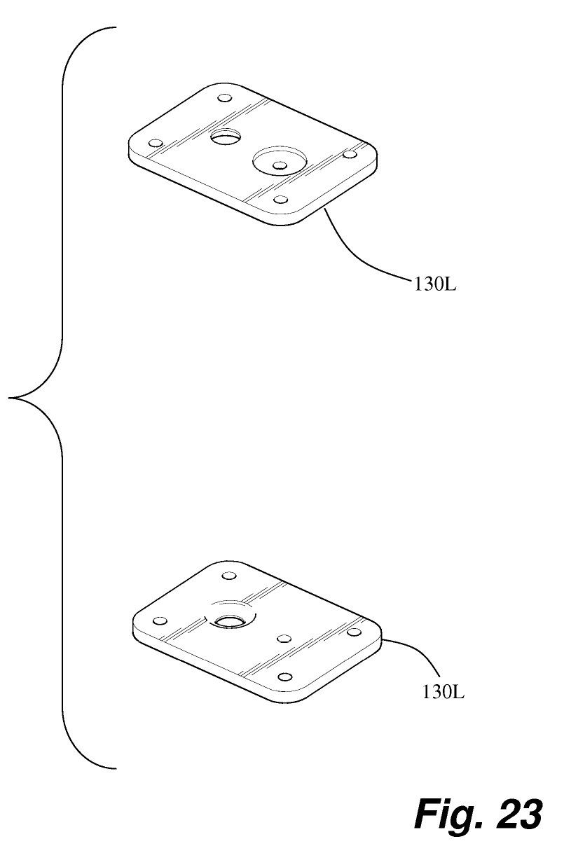

FIG. 23 shows opposite side views of a left bladder plate 130L. For orientation see FIG. 2. The left bladder plate 130L fits between the left bladder 140L and the vertical section 220L of left L-bracket 110. A right bladder plate 130R fits between the right bladder 140R and the vertical section 220R of right L-bracket 170. The right bladder plate 130R is essentially a mirror image of the left bladder plate 130L.

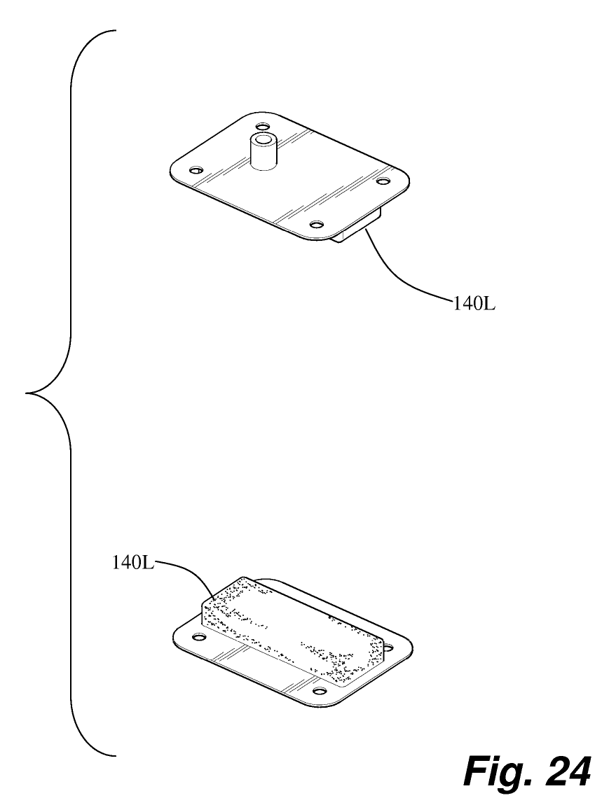

FIG. 24 shows opposite side views of left side bladder 140L. The right side bladder 140R is essentially a mirror image of the left side bladder 140L.

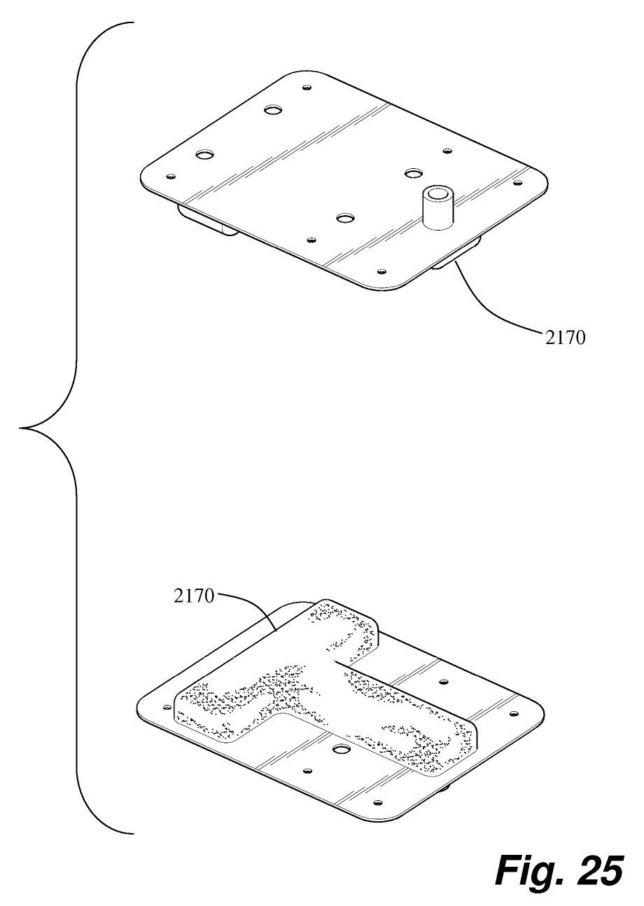

FIG. 25 shows opposite side views of the top bladder 2170. The top bladder 2170 is orientated and positioned as shown in FIG. 2. The top bladder 2170 has an overall T-shape, but it should be understood that the top bladder 2170 can have any suitable overall shape and is explicitly not limited to a T-shape.

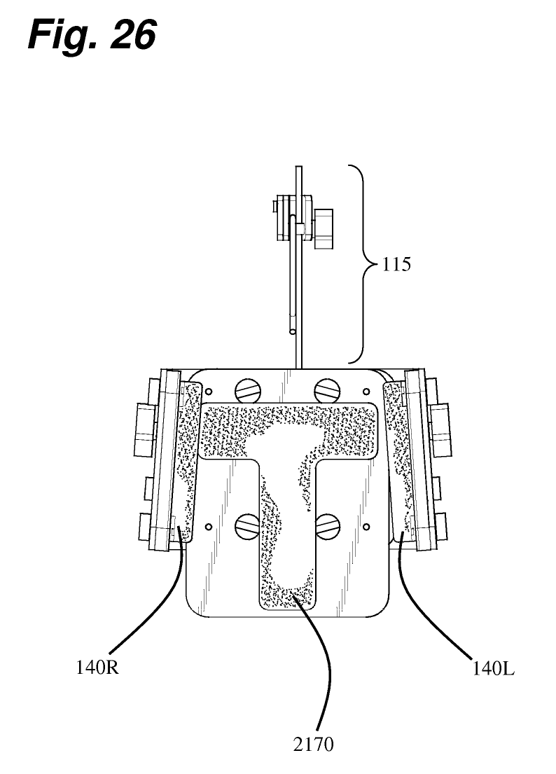

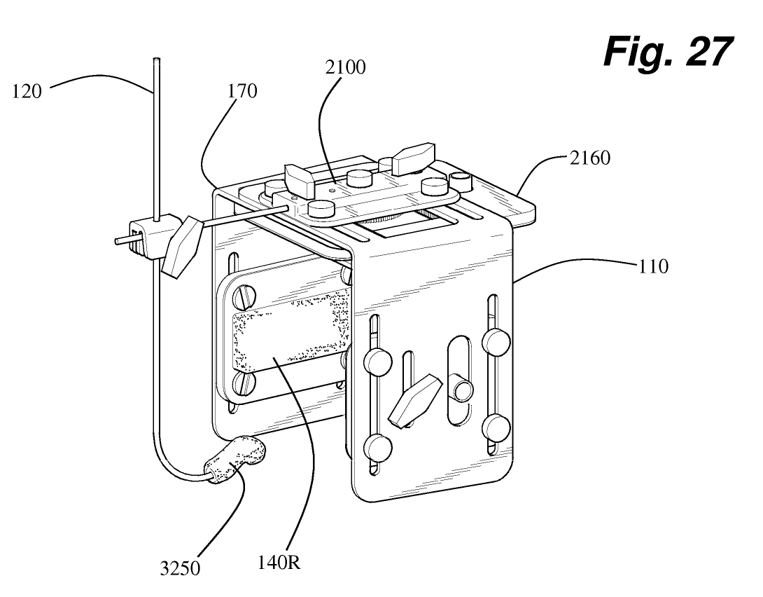

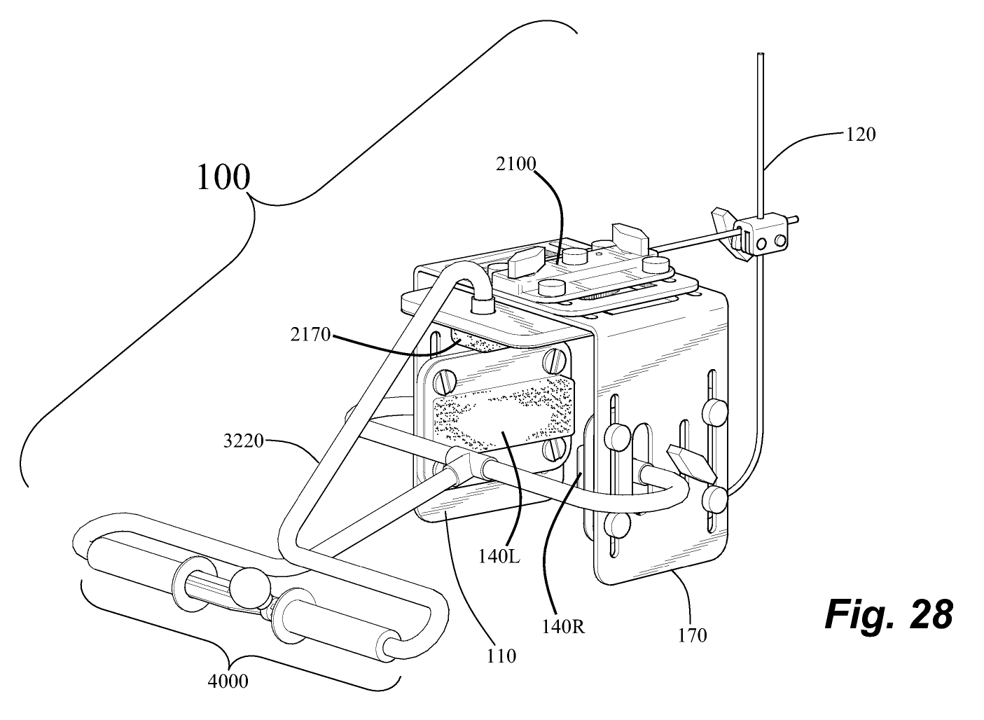

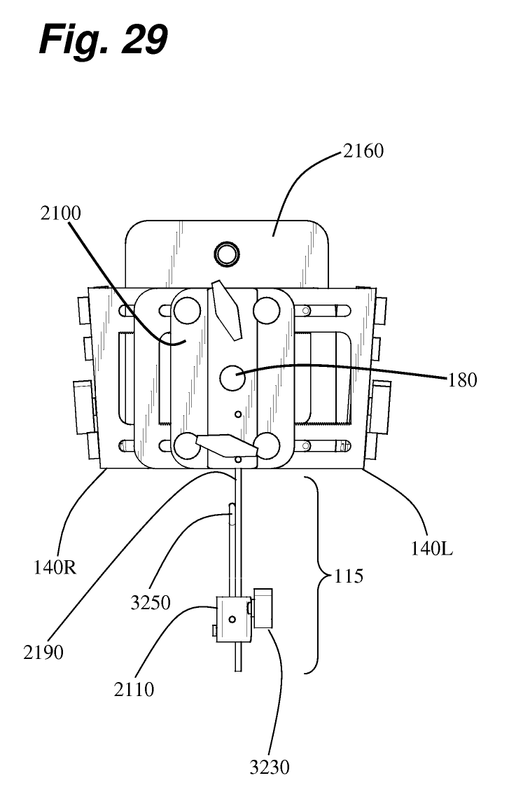

FIGS. 26 through 29 respectively show bottom, perspective front, perspective rear, and top views of the cranial apparatus 100. The tubing 3220 and pump system 4000 is only shown in the rear perspective view of FIG. 28.

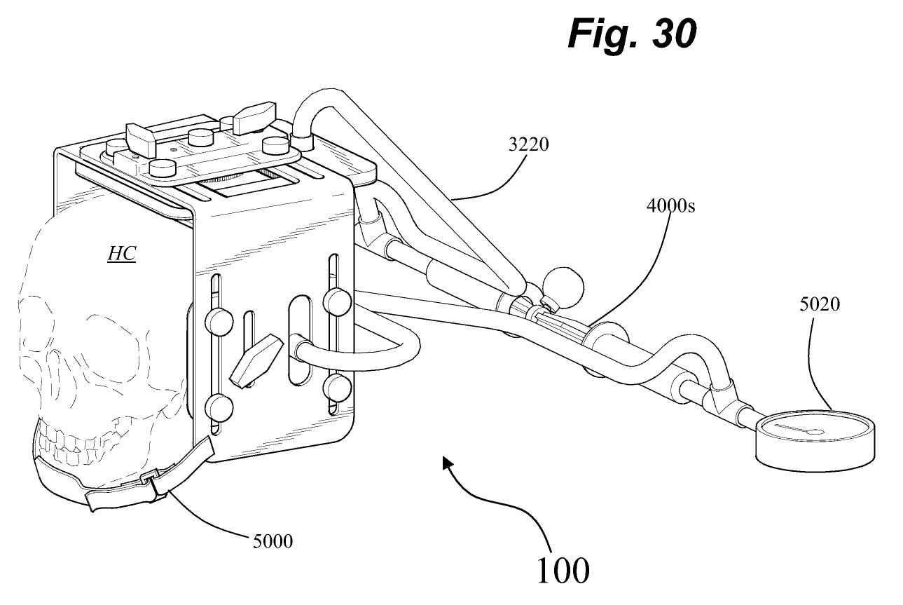

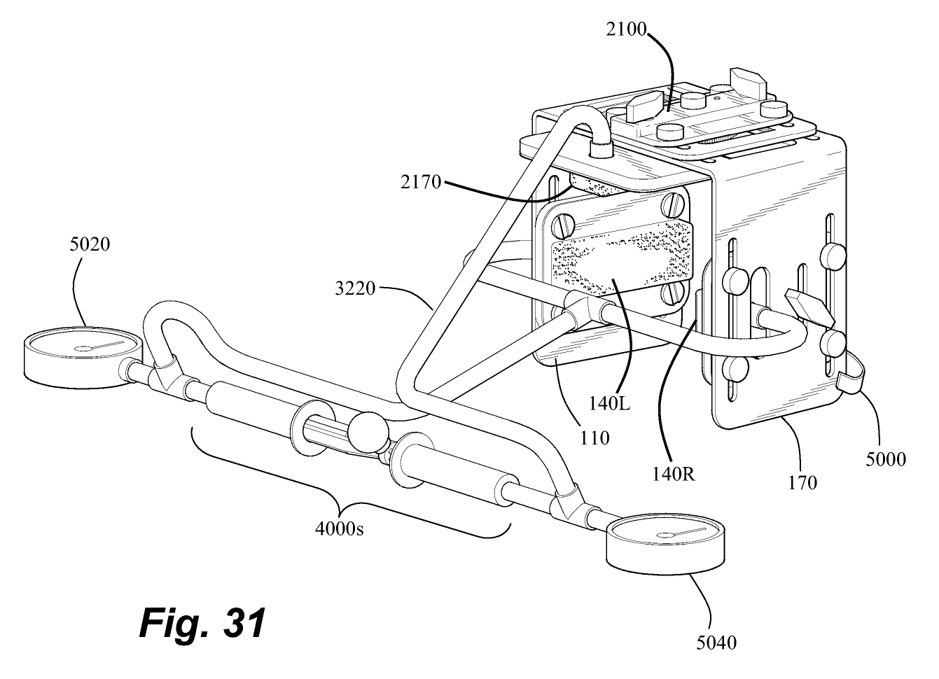

FIGS. 30 and 31 respectively show front and rear views of an embodiment in which a chinstrap 5000 is fitted to the cranial device 100. FIG. 31 shows first and second pressure gauges 5020 and 5040 fitted to the opposite ends of syringe pump 4000s.

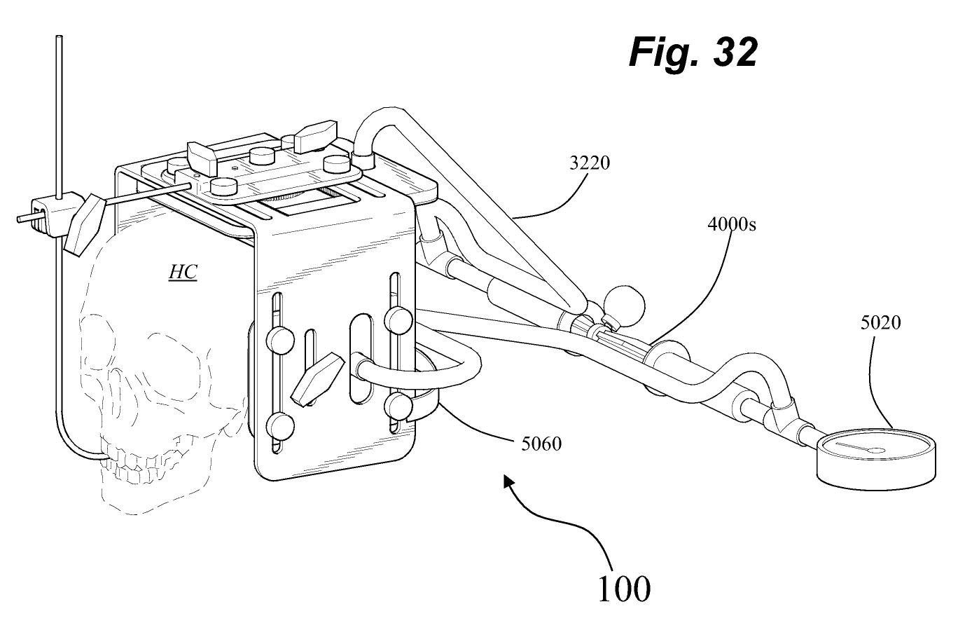

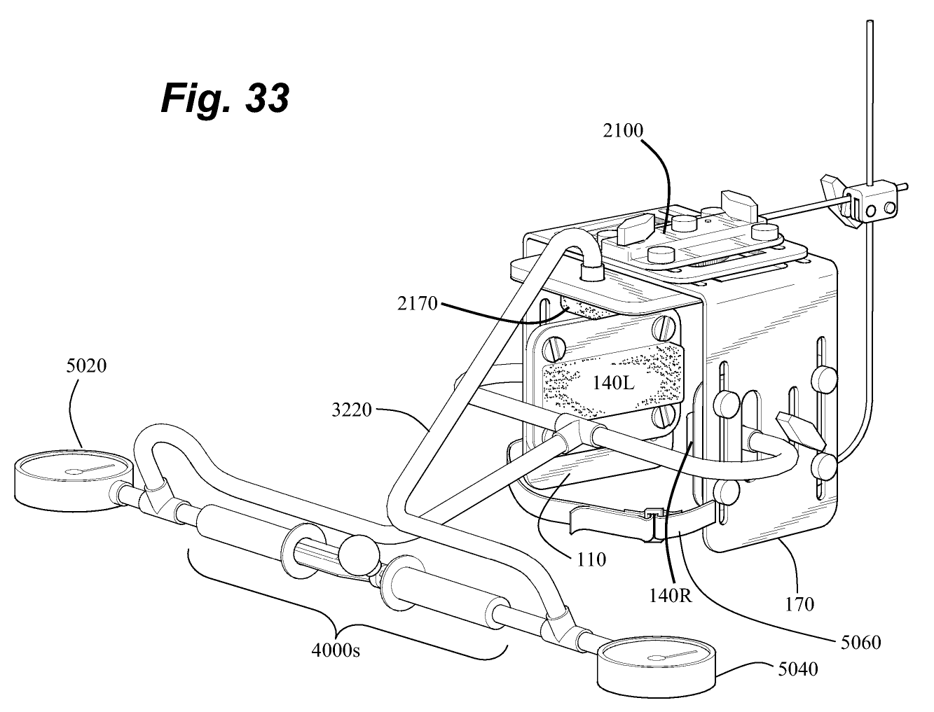

FIGS. 32 and 33 respectively show front and rear views of an embodiment in which a rear-neck strap 5060 is fitted to the cranial device 100.

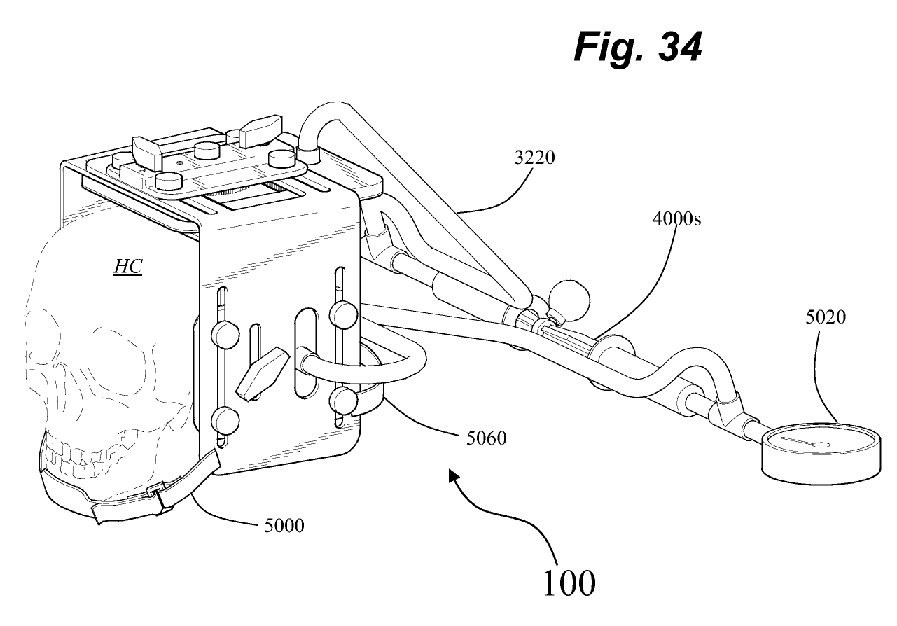

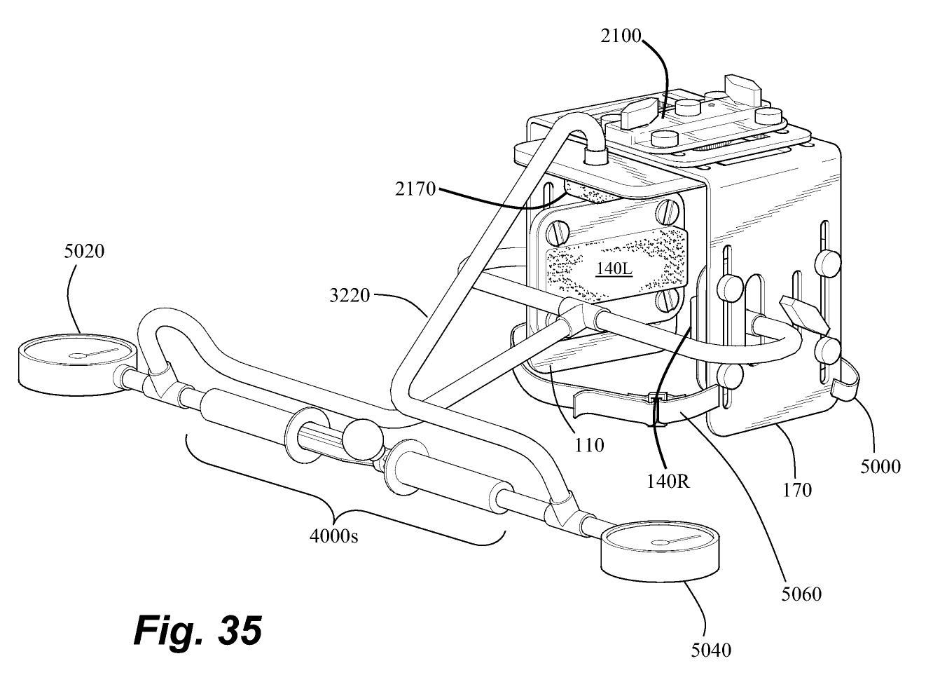

FIGS. 34 and 35 respectively show front and rear views of an embodiment in which a chinstrap 5000 and a rear-neck strap 5060 are fitted to the cranial device 100.

The straps 5000 and 5060 can be made out of any suitable material such as synthetic material (e.g., plastic) or a natural material such as leather. The opposite ends of each strap can be attached by any suitable means, e.g., by gluing the ends of each strap to the left and right L-brackets 110 and 170. Alternatively, fasteners can be used such as, but not limited to, rivets. Alternatively, suitably positioned apertures can be formed in either or both L-brackets 110 and 170 and the ends of the straps 5000 and 5060 affixed to the apertures (not shown).

In one embodiment the weight of the cranial device 100 can be supported, for example, by an extension (not shown) between a patient's treatment bed (not shown) and the back of the cranial device 100. Alternatively, the cranial device 100 can be suspended by any suitable means. Supporting the weight of the cranial device 100 helps avoid adding a constant low-level compression to the saggital suture area of a patient's skull. In this embodiment the cranial device 100 can be used as follows: gentle short duration pressurization on top bladder 2170 at pressure of 3-5 oz/square inch (while the side bladders 140L and 140R are depressurized), followed by a longer pause (non of the bladders pressing against the cranium) followed by side bladders 140L and 140R pressurized at about 3-5 oz/square inch, followed by a longer pause. Then, the steps repeated multiple times. In this embodiment the patient is encouraged to have deep rhythmic breathing during the treatment.

The invention being thus described, it will be evident that the same may be varied in many ways by a routineer in the applicable arts. Such variations are not to be regarded as a departure from the spirit and scope of the invention and all such modifications are intended to be included within the scope of the claims.

I claim:

1. A cranial apparatus for applying pressure to a patient's skull, the patient's skull being made up of cranial bones interconnected by cranial sutures, the patient's skull having right and left sides and a top side, said cranial apparatus comprising:
a means for providing left and right reversible inflation against the left and right sides of a patient's skull, said means for providing left and right reversible inflation comprise at least one reversibly inflatable right side bladder and at least one reversibly inflatable left side bladder;
a means for providing opposing left and right counter forces in the x-axis, said means for providing opposing left and right counter forces respectively comprising opposite left and right L-brackets, each of said left and right L-brackets having a horizontal section and a vertical section;
a means for providing downward pressure in the y-plane against the top of a patient's skull, said means for providing downward pressure in the y-plane against the top of a patient's skull comprising at least one top bladder, wherein said at least one bladder is positioned parallel to and below said left and right horizontal sections of said left and right L-brackets;
a means for providing a counter-force in an upwards direction in the y-axis; and
a means for providing working fluid to said at least one top-bladder and said at least one left bladder and said at least one right bladder, wherein said means comprises a reversible flow pump system coupled to tubing for delivering working fluid to reversibly inflate in desired sequence the at least one top bladder, the at least one left side bladder and the at least one right side bladder.
